(19) United States
(12) Patent Application Publication
(10) Pub. No.: US 2015/0217122 A1
(43) Pub. Date: Aug. 6, 2015

(54) MEDICAL DEVICE FOR HEART STIMULATIONS

(71) Applicants: James Andrew LESKOSEK, Summerland (CA); Ted Simonian KORDOLIA, Nanaimo (CA)

(72) Inventors: James Andrew LESKOSEK, Summerland (CA); Ted Simonian KORDOLIA, Nanaimo (CA)

(21) Appl. No.: 14/612,038

(22) Filed: Feb. 2, 2015

Related U.S. Application Data
(60) Provisional application No. 61/934,293, filed on Jan. 31, 2014.

(51) Int. Cl. A61N 1/39

(52) U.S. Cl. CPC A61N 1/3975; A61N 1/3987

(57) ABSTRACT

The present invention is a heart stimulation medical device that is electrically self-sustaining. The present invention uses the patient's body as a battery or source of electric current. The device relates to the functions of a pacemaker and detecting and/or treating cardiac arrest. The present invention may be characterized as at least including a heart sensor for detecting a heartbeat of the patient and for generating a heartbeat signal; a processor for receiving the heart beat signal and for transitioning the medical device between an active state and an inactive state; an electrical pulse output that is electrically connectible to a portion of the patient's heart; and a power harvester that is adapted to harvest electric current from the patient's body and the power harvester is electrically connected to at least the pulse output and the processor so that the medical device is electrically self-sustaining when in an active state.

### FIELD OF THE INVENTION

The present invention relates to medical devices for implanting in patients. In particular, the present invention relates to medical devices for implant in a patient's heart for monitoring and stimulating the patient's heart.

### BACKGROUND OF THE INVENTION

Known medical devices and systems that are used for in situ and in vivo treatment and monitoring of the human heart are typically large and bulky. The known medical devices and system require batteries to power various components including, at least, logic systems, sensors and electrodes. The batteries present a risk that they will corrode within the body of a medical device recipient and expose the recipient to toxic components of the battery. Such corrosion can cause serious health repercussions to the recipient. Batteries also have a limited reserve of power. If the medical device loses power, the device becomes inoperable and the recipient may at a greater risk for a cardiac event, for example cardiac arrhythmia and/or cardiac arrest. As the power reserve depletes over time, the battery must be replaced or recharged by a surgical intervention. Inherently, any surgical intervention is associated with risks and complications that can be detrimental the recipient's health.

Some known medical devices for in situ and in vivo treatment and monitoring of the human heart include components, for example hard circuits, which are awkwardly shaped so as to cause protrusions on the surface of the recipient's body. Furthermore, hard circuits are fragile and they can become damaged if subjected to a sufficient impact. Damaged hard circuits also typically require a surgical intervention to replace the damaged components so that the medical device may resume normal operations. As described above, the surgical intervention in turn exposes the recipient to further risks and complications.

The known medical devices for in situ and in vivo treatment and monitoring of the human heart systems are also commonly very expensive due to high manufacturing costs and the use of expensive materials.

### SUMMARY OF THE INVENTION

The present invention is a heart stimulation medical device that requires no battery as it is electrically self-sustaining. Instead, the present invention uses the body of a live patient that has received the medical device as a battery or source of electric current. The device relates to the functions of a pacemaker and detecting and/or treating cardiac arrest. Typically, a pacemaker, also referred to as implantable cardioverter-defibrillator ICD, is a battery-operated device that is implanted within a patient's body. The pacemaker's battery may comprise toxic components and it is typically bulky. The pacemaker may create a lump that patients can feel on their chest, which can be uncomfortable and may agitate some patients. Such pacemakers comprise wires that directly enter into the patient's heart, these wires can become faulty and require replacement surgery. The patients with battery-operated pacemakers typically require surgery every 5-7 years to replace the battery within the device.

The present invention electrically stimulates the heart only when the patient's heart is not beating and there is no pulse. The present invention may work in-conjunction with pacemakers and ICD's so as to not interfere with the operation of those devices for detecting and treating dysrhythmias, arrhythmias, and/or other cardiac events.

The present invention comprises a device that uses a circuit that is very small and thin compared to other implantable medical devices. The device has attached to it a relatively small power generator as a power a source. The device's power generator constantly gathers electrons, electricity and/or electric current from the patient's body that can be used as a source of power for the device. The device preferably includes a sensor to sense for any external electricity, electric current or electric shock that may be passing through the patient's body. The power generator harvests the electric current from the flow of charged particles within the patient's body such as ions or electrons from cells, and/or nerve clusters in the patient's body.

In one embodiment of the present invention, the device does not include a battery. In a second optional embodiment of the present invention, the device does include a battery that is used as a secondary power source.

The device comprises a heart sensor for detecting the patient's heartbeat. The terms “pulse” and “heartbeat” are used interchangeably herein. If the heart sensor detects a regular heartbeat or an abnormal heartbeat, the device remains in an inactive state. While in its inactive state, the electric current that is collected by the power generator is grounded back into the patient's body, so that none of the generated electricity is used. If the pulse sensor detects a lack of a heartbeat or pulse, the device is put into an active state. When the device is in the active state, the electricity generated by the power generator is allowed to flow through or into the device. This flow of electricity charges the device so the device can start stimulating the heart to resume rhythmic contractions. When the heart sensor of the device has detected an absence of a heartbeat, the device will electrically stimulate the patient's heart. For example, the device may stimulate one or more of the patient's cardiac conducting system, possibly including the sinoatrial node (SA), the patient's myocardial contractile cells or the patient's cardiac nervous tissue. The stimulation from the device can be at a set timed interval and at a predetermined amperage so as to depolarize one or more portions of the patient's heart to stimulate contractions of the cardiac contractile tissue. This stimulation may cause the patient's heart to resume rhythmic contractions and cause blood to resume flowing throughout the patient's circulatory system.

Optionally, the device will remain in the active state until the device detects one of three events, after which the device will return to the inactive state. The three events are: (1) if the device detects a higher shock of electricity than the amperage that the device is generating, in this case the device will revert back into its inactive state (that is, presumably patient has received some form of external electrical stimulation, for example by an external electric defibrillator); (2) the heart has resumed rhythmic contractions due to the device's stimulation, and (3) the device's power generator can no longer generate electric current from the patient's body, and thus the device can no longer function.

The device addresses, for example, instances where the patient experiences a cardiac event where their cardiac rhythm is disturbed, also referred to as arrhythmia, and the patient is not treated with a defibrillator quickly enough to avoid cardiac arrest. Arrhythmia and cardiac arrest, which are associated with risks of suffering brain damage, from a lack of oxygen and glucose delivery to the brain, and patient death. Thus, at least in one aspect, the device may allow more time for a patient whose heart has stopped beating to receive treatment in an effort to regain a normal cardiac rhythm.

In summary, the present invention may be characterized in one aspect as at least including:
a) a heart sensor for detecting a heartbeat of the patient and for generating a heartbeat signal;
b) a processor for receiving the heart beat signal and for transitioning the medical device between an active state and an inactive state;
c) an electrical pulse output that is electrically connectible to a portion of the patient's heart; and
d) a power harvester that is adapted to harvest electric current from the patient's body and the power harvester is electrically connected to at least the pulse output and the processor so that the medical device is electrically self-sustaining when in an active state.

The processor regulates an output pulsing rate of the electrical pulses from the pulse output, upon detection of any pulse or beating by the heart sensor the device is deactivated so as to cease the output of the electrical pulse, while continuing said sensing by the heart sensor.

In another aspect, the present invention may be characterized as a medical implant device, which includes:
a) a heart rate sensor implant adapted to be implanted into a live human body in cooperation with the heart within that body so as to detect a rate of pulsing of the heart;
b) a processor implant adapted to be implanted into the live human body and cooperating with the heart rate sensor implant to monitor the pulsing of the heart and to detect when there is no pulsing of the heart; and,
c) an energy harvesting implant, wherein the energy harvesting implant is adapted to be implanted in the live human body for electrically conductive coupling to an internal, electricity or electric current producing portion of the live human body so as to collect and transmit an electric current derived therefrom so that the medical implant device is electrically self-sustaining.

In another aspect, the present invention may be characterized as a method of using the medical implant device, the method including at least the steps of:
a) providing the medical implant device;
b) implanting the medical implant device;
c) establishing an electrical connection between the implanted medical implant device and a heart of a recipient of the implanted medical device implant; and
d) collecting electric current from the recipient so as to make the medical implant device electrically self-sustaining.

The processor implant is in electrical communication with the energy harvesting implant so as to receive, and be powered by, the electrical current from the energy harvesting implant.

A circuit is associated with the processor implant and cooperates therewith. The circuit has a discharge node and is adapted to store an electrical charge from the electrical current.

When the heart rate sensor implant detects any pulsing of the heart the processor implant biases the circuit into an inactive state, and, when the heart sensor implant detects an absence of pulsing, the heart sensor implant biases the circuit so as to discharge the electrical charge to the discharge node so that, when the discharge node is electrically connected to an electrical impulse receiving node on the heart, the electrical charge is discharged to the receiving node on the heart.

Advantageously the device 10 may be flexible and/or mounted on a flexible substrate. In one preferred embodiment, which is not intended to be limiting, the circuit includes at least one relay, and in one preferred embodiment, the circuit includes a series of relays. The relays may provide the timing and/or logic, and may thus themselves comprise the processor. The relays are reactive to a signal from said processor that indicates there is an absence of a heartbeat, i.e., cardiac arrest, so that the electrical discharge to the heart is in response to the detected cardiac arrest.

The relays may advantageously be micro-electrical mechanical devices or so-called MEMS devices, or are incorporated into MEMS devices. The relays are thrown by electricity or electric current harvested from the electrical nodes in the human body. The harvesting may be done by harvesting material, for example semi-conductor material mounted so as to cover, or be embedded in, or be melded with or otherwise electrically coupled to one or more of the electrical nodes in the body. The electrical nodes may be cells or nerve clusters, for example. The use of the circuit and harvesting material in such a manner thus removes the need for a battery in such devices, or removes the need for a primary battery in embodiments that may use a battery as a secondary power source.

### BRIEF DESCRIPTION OF THE DRAWINGS

Various examples, embodiments, options and aspects of the present invention are described in detail below, with reference to the accompanying drawings. The drawings may not be to scale and some features or elements of the depicted examples may purposely be embellished for clarity. Similar reference numbers within the drawings refer to similar or identical elements. The drawings are provided only as examples and, therefore, the drawings should be considered illustrative of the present invention and its various examples, embodiments, options and aspects. The drawings should not be considered as limiting or restrictive to the scope of the invention.

FIG. 1 is a diagrammatic view of a device according to one aspect of the invention having a circuit employing relays for timing of electrical pulses to the SA node.

FIG. 2 is in perspective view, one embodiment of the device of FIG. 1 coupled to a flexible substrate for mounting into a patient's body.

FIG. 3a is a diagrammatic flow chart of the operation of the heart sensor.

FIG. 3b is a diagrammatic flow chart of the operation of the power harvesting.

FIG. 3c is a diagrammatic flow chart of the operation of the defibrillator sensor.

FIG. 4 is a diagrammatic flow chart of the operation and use of one embodiment of the invention.

FIG. 5a is a top plan view of one layer of electrical lines that may be used as part of an example circuit for use with the device of FIG. 1.

FIG. 5b is a top plan view of another layer of electrical lines that may be used as part of an example circuit for use with the device of FIG. 1.

FIG. 5c is a top plan view of another layer of electrical lines that may be used as part of an example circuit for use with the device of FIG. 1.

FIG. 5d is a top plan view of an example circuit that may be formed by the electrical lines of FIGS. 5a, b and c.

### DETAILED DESCRIPTION OF EMBODIMENTS OF THE INVENTION

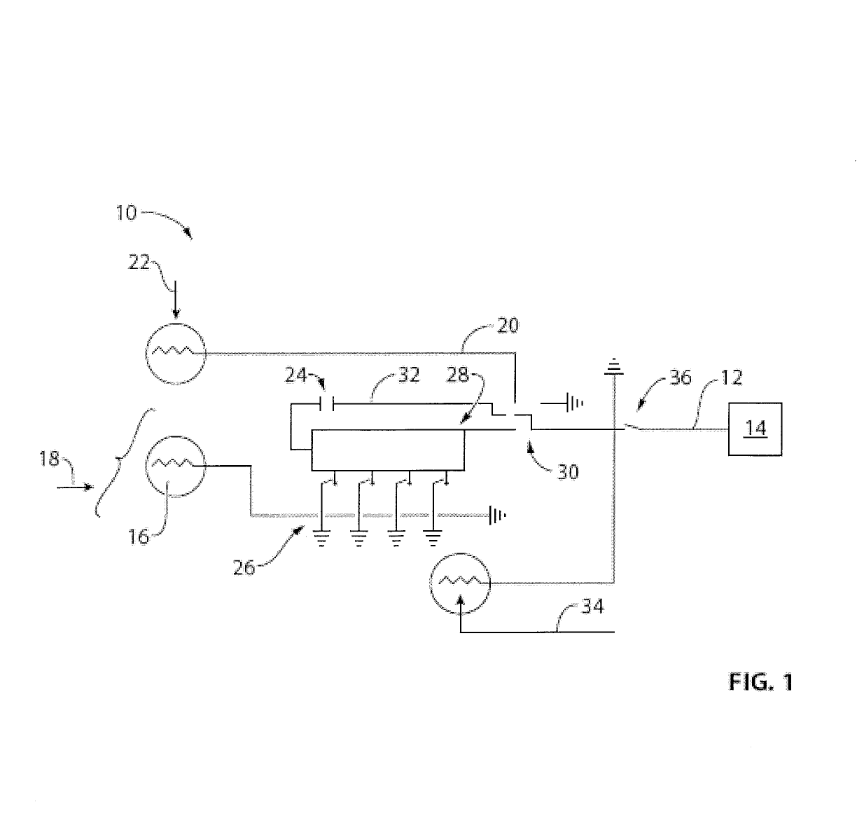

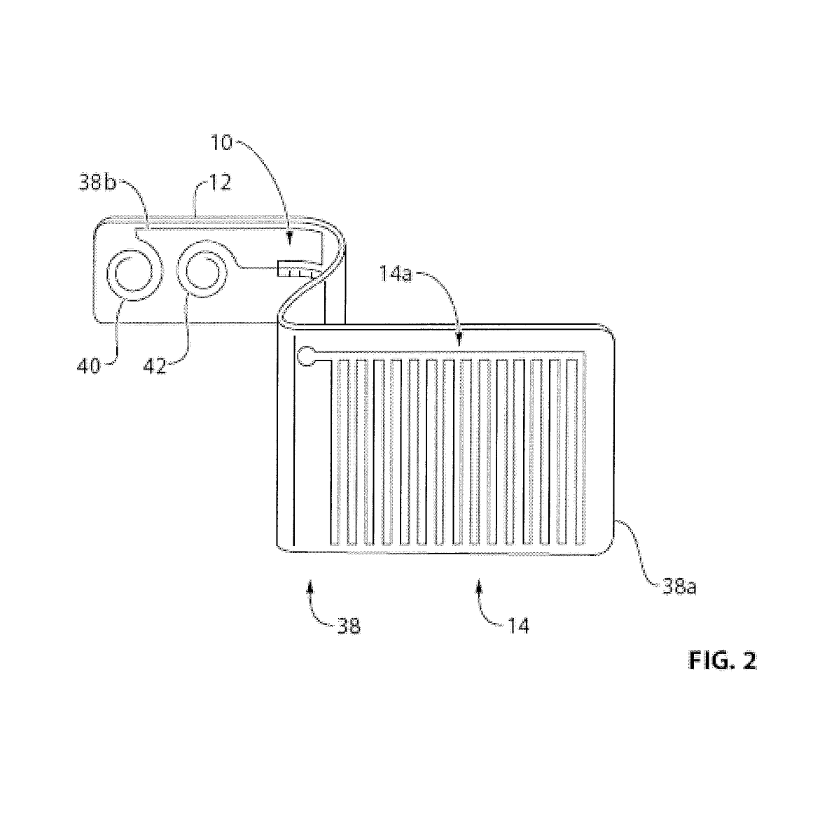

With reference now to the Figures, in FIG. 1 it may be seen that heart stimulation device 10 comprises a power line 12, which provides electrical communication between various components of the heart stimulation device 10 and an electrical harvesting device 14. Power line 12 conducts harvested electrons, electricity and/or electric current. The power line 12 is extremely conductive, for example, the power line 12 may be a super-conductor. The harvesting device 14 may include a collective field 14a, which attaches to the surface of a nerve cluster (not shown) in the body a patient that received the device 10 as an implant. A harvesting device 14 is shown in FIG. 2 by way of example. As will be known to those in the art, various materials may be used for the collective field 14a to attract electrons, electricity and/or electric current from the nerve cluster. Such materials may include semi-conductors. The harvesting device 14 may pluck electrons off a given area and transfers those electrons through the circuits to a grounded area, such as oxygen molecules. Electric current harvested by harvesting device 14 is conducted along power line 12 to power the device 10. The electric current that is harvested by the harvesting device 14 powers the device 10 and allows the device to operate without a battery power source. In this regard, the device 10 is electrically self-sustaining.

A heart sensor 16 within device 10 monitors a patient's heartbeat, which is also referred to herein as a pulse. FIG. 1 depicts the pulses as a heartbeat input 18. For example, the heart sensor 16 may detect changes in the electrical properties of the SA Node and the heartbeat input 18 may reflect the rhythmic depolarization of the SA Node. Alternatively, the heart sensor 16 may detect changes in the electrical properties, or other properties, of other portions of the heart, such as other portions of the conducting system and/or the cardiac contractile tissue. If heart sensor 16 does not detect a current/electrical pulse from heartbeat input 18 within a set time, this may indicate that the patient has had a cardiac arrest. If the heart sensor 16 detects a cardiac arrest, an electrical charge is delivered by output line 20 to the pulse output device 22, which may also be referred to as a pulse generator, so as to stimulate the heart of the patient at the Sino-Atrial (SA) node. Alternatively, the pulse output device 22 may deliver a threshold amplitude of electric current to other portions of the patient's heart so that the myocardial contractile tissue will contract. For example, the pulse output device 22 may deliver a threshold amplitude of electric current to one, or more, or all of the patient's cardiac conducting system, the myocardial contractile tissue and portions of the patient's cardiac nervous system for example the sympathetic cardiac nerves.

The charge pulse is stored within device 10 within, for example, capacitors 24. Capacitors 24 trip upon the absence of a heartbeat input 18 as detected by heart sensor 16 so as to throw, also referred to as switch or actuate, the loop relay, switching line 12 to 20 delivering the charge pulse, for example in the form of electric current, to the pulse output 22.

Device 10 also may include a counting mechanism 28, which may for example be accomplished by the use of capacitors or other counting means as would be known by someone skilled in the art. Counting mechanism 28 provides that a charge built up in capacitor 24 is discharged by switching power line 12 to pulse output 22 at a predetermined, regular and a governed frequency that is suitable to stimulate the patient's heart.

Loop relay 30 is provided so as to switch between power line 12, a trigger mechanism line 32 and the output line 20.

A surge sensor 34 is provided to detect an electrical shock or electric current surge that is greater than a pre-set level. The pre-set level is such that the detected electric current surge may indicate that the patient is being treated by defibrillation. If the surge sensor 34 detects an electric current surge, presumably from a defibrillator, then the power line 12 is disconnected from device 10 including the pulse output 22, for example, by means of an actuatable relay 36.

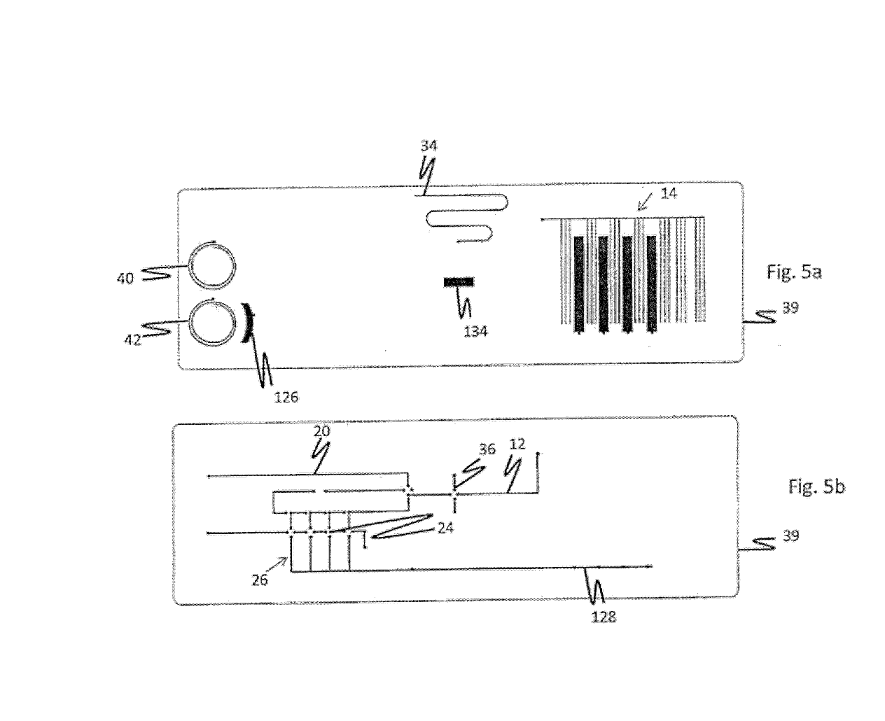

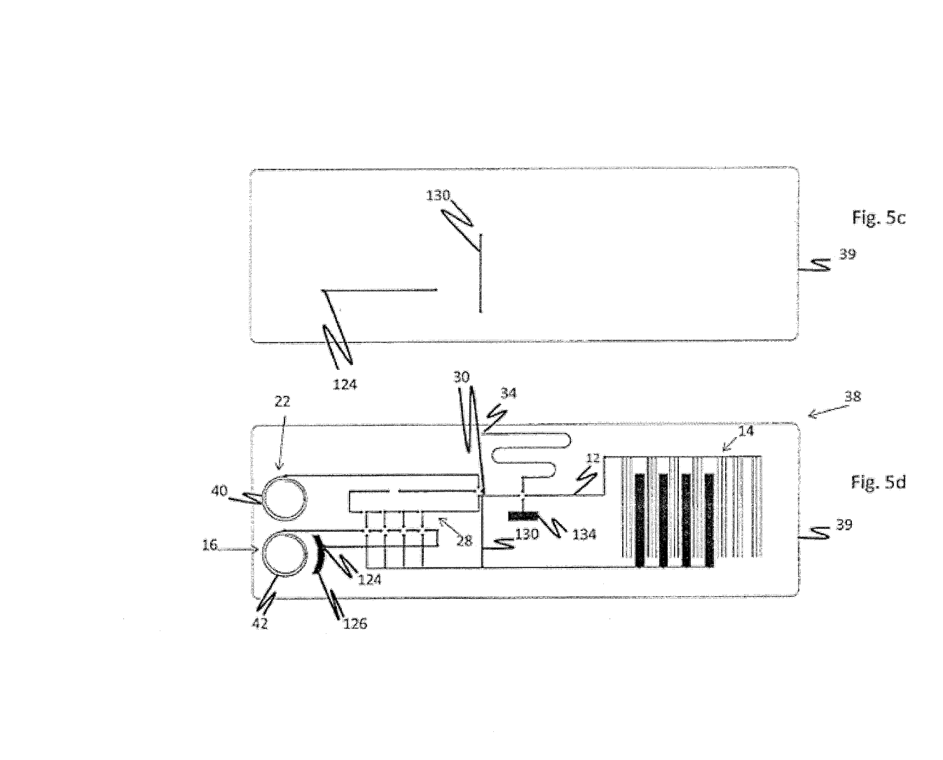

In the embodiment of FIG. 2, the harvesting device 14, and in particular collective field 14a, may be formed at one end 38a of flexible device 38. The flexible device 38 may be any form or shape of flexible member, sheet, film, band, etcetera that is adapted to extend from the electron harvesting site to near to or at the SA node. In the opposite end 38b are mounted for example coil 40, which may provide an electrical connection to the SA Node, and coil 42 which provides the electrical sensory input for heart sensor 16. The device 10 circuit of FIG. 1 may be mounted in or on the flexible device 38, for example between the ends 38a and 38b. In one embodiment, not intended to be limiting, device 10 and its associated electrical lines are mounted inter-leaved between flexible adjacent layers 39 forming the flexible device 38. FIGS. 5a, b and c depict three example electrical line layouts that can each be printed on, or embedded in, on an individual flexible layer 39. Optionally, the electrical lines of the device 10 may be super-conductors.

FIG. 5a depicts, among other things, a ground 126 for the relay 26 or the capacitor 24 and a ground 134 for the surge sensor 34. FIG. 5b depicts, among other things, a ground connection 128 for the counting mechanism 28. FIG. 5c depicts, among other things, a connection line 130 for connecting the loop relay 30 to the ground connection 128 and a connection line 124 to connect the capacitor 24 to the ground 126.

The flexible layers 39 are then positioned adjacent each other, for example on top of each other, to form the planar circuit of the flexible device 38, as depicted in the example of FIG. 5d. For example, the flexible layer 39 depicted in FIG. 5a may be the top layer, and the flexible layer 39 of FIG. 5b may be the middle layer and the flexible layer 39 of FIG. 5c may be the bottom layer. Alternatively, the flexible layer 39 of FIG. 5b may be the bottom layer and the flexible layer 39 of FIG. 5c may be the middle layer.

For the portions of the device 10 that require non-corrosive and non-reactive materials, such as the flexible layers into which the electrical lines are printed or embedded, the following materials may be suitable, but are not limited to: medical grade polymers, medical grade silicone and vitamin E coated, or infused, polyethylene of various weights.

Optionally, the device may comprise nanowires, biological nanowires, biofilm from bacteria, and combinations thereof. Nanowires can be used as the power harvesters or the ground for the device 10. This is similar to how Shewanella oneidensis soil bacteria or other types of common soil bacteria use nanowires to push electrons to a ground to produce electric current. The harvester device 14 and the ground can also comprise nanocrystals or quantum dots, which may increase the performance of the device 10. Use of gold nanoclusters, gold nanoparticles, platinum nanoclusters and platinum nanoparticles, with or without carbon, may also increase the performance of the device 10. The expression “performance of the device” refers to at least the ability of the device 10 to efficiently and quickly harvest electric current from the patient's body and the speed and amperage at which the harvested electric current travels through the electrical lines of the device 10.

As stated above, the device may use relays. This is not to limit the device only to the use of only relays as other mechanical/electronic means may be employed for timing, logic, and etcetera, as would be known to one skilled in the art. The reason that relays are preferred is that relays are reactive in the sense that they are not using power all the time to run the normal function of the device. This means that the power from the body's normal electrical functioning may work in unison with the device 10. In the embodiment of FIG. 1, the energy that the body produces turns over small relays 26 (switches) that can be micrometer size or smaller, to run the logic. Thus, the device 10 may be reactive to the body's electrical signal, rather than proactive. The lack of a heart beat input 18 signal from the heart causes the electric current that is being harvested from the body to throw the device relays or switches to send the charge pulse to the patient's heart for the predetermined time at a predetermined amperage. This eliminates the need for a battery for powering the device's logic. Thus relays 26 may be preferred for the device, or other switches that operate reactively.

The collective field 14a of the device 10 may attract electrons, or other charged particles, from cells or nerve clusters within the patient so as to collect electric current. The device 10 may comprise a material, such as a semi-conductor material, that attracts electrons from part of the collective field. A secondary type of a power generation system may also be added to the device 10. For example, a battery may be provided for use as a secondary, or backup, power source. Preferably, a battery is only used as a secondary power source.

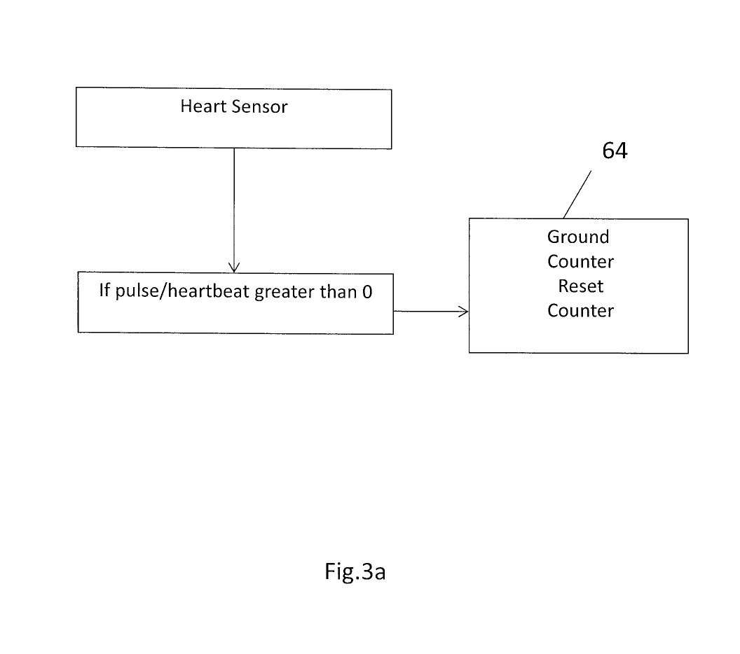

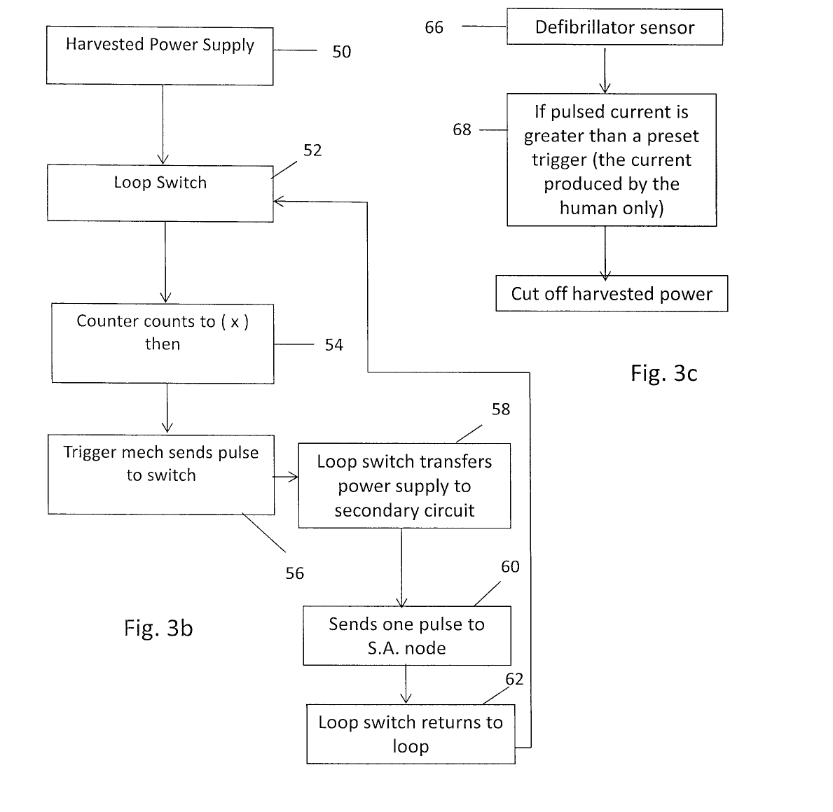

As seen in FIG. 3a, the heart sensor 16 detects all heartbeat input 18 signals from the patient's heart. The counting mechanism 28 is continually reset unless the heart sensor 16 does not detect the heartbeat input 18. In FIG. 3b, the logic of counting mechanism 28 is diagrammatically set out. The harvested power supply harvested at step 50 is transmitted, for example by the power line 12, into a loop switch 52, which for example, may be loop relay 30 seen in FIG. 1. Within counting mechanism 28, the counter counts to a pre-set time or other variable (shown in step 54 as variable x). Upon the counter achieving the pre-set count and not receiving input from the heart sensor 16 that a heartbeat input 18 was detected, then, in step 56, a trigger mechanism such as capacitor 24 shown in FIG. 1 sends a stored electrical pulse to the loop relay 30. The device 10 is now in the active state. In step 58, the power supply is transferred to a secondary circuit such as the electrical output line 20 in FIG. 1. In step 60, the electrical charge pulse is sent to the SA node, such as via pulse output 22 in FIG. 1 or SA node coil 40 seen in FIG. 2. In step 62, the loop switch returns control to step 52 so as to re-start the count in step 54, which has been reset in the counter reset step 64.

As seen in FIG. 3c, concurrent with the operation of the heart sensor 16 of FIG. 3a, and the power pulse loop of FIG. 3b, in FIG. 3c defibrillator sensor 66 constantly monitors for an electrical shock or electric current surge that exceeds a predetermined amount. Detection of such an electrical shock or electric current surge indicates that treatment by a defibrillator has likely begun. Detecting an electrical shock or an electric current surge that exceeds the predetermined amount will trigger the device 10 to return to the inactive state. In step 68, if the electrical shock or current surge detected in step 66 is greater than a preset value, wherein advantageously the preset trigger may be the amount of current produced by the human body, then the harvested power from the electrical harvesting device 14 that is traveling along power line 12 may be cut off, for example by throwing relay 36.

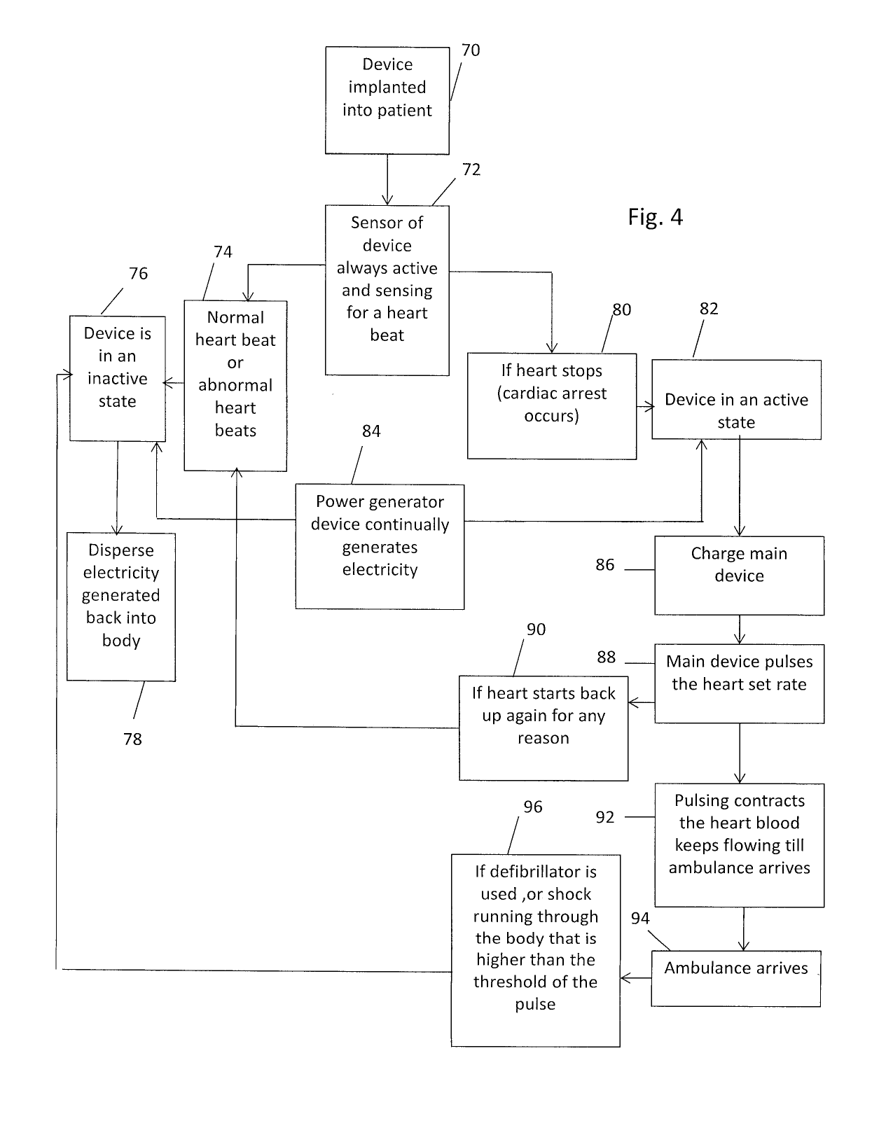

An overall flow chart of the use of the present device 10 and the logic the processor uses is depicted in FIG. 4. In step 70, the medical device 10 according to the present invention is implanted into a patient so as to harvest electrical power from the patient's body, which thereby provides electrical power to the heart sensor 16 of the device 10 for monitoring the patient's heartbeat. Thus in step 72 the heart sensor 16 is always actively monitoring the patient's heartbeat. In all cases other than upon cardiac arrest, as seen in step 74 the device 10 according to the present invention is maintained in its inactive state in step 76. While in its inactive state excess electrical power is dispersed or grounded back into the patient's body in step 78.

If in the sensing step 72 the heart sensor 14 detects no pulse or heartbeat, indicating that the patient has had a cardiac arrest, as determined in step 80, then the processor switches the device into the active state in step 82. As described above, no matter if the device is inactive as in step 76 or active as in step 82, the harvested electrical energy is provided as power to the device continually as set out in step 84. Following activation of the device in step 82, the harvested electrical power is used to charge the stored electrical pulse in step 86.

In step 88 circuit 10 of the device pulses the patient's heart at a predetermined rate. In step 90, if the sensor detects that the patients heart has started to beat, that is, any pulse is detected in the patient, in step 90 then the processor logic transfers to step 74 described above. Until a heart re-start is detected the pulsing in step 88 continues contracting the patient's heart in step 92 until paramedics arrive in step 94 and a defibrillator is used by the paramedics in step 96, which is then detected by the defibrillator sensor such as indicated at step 66 in FIG. 3c, whereupon the processor logic is transferred to step 76 and the device is inactivated.

While reference is made above to paramedics using a defibrillator, it is understood that a patient may receive a defibrillation treatment by a non-paramedic, for example by an untrained person using an automated, external defibrillator (AED). An AED will also be detected by the defibrillator sensor to transition the device to the inactive state.

While the above disclosure describes certain examples, embodiments, options and aspects of the present invention, various modifications to the described examples, embodiments, options and aspects will also be apparent to those skilled in the art. The scope of the claims should not be limited by the examples, embodiments, options and aspects provided above; rather, the scope of the claims should be given the broadest interpretation that is consistent with the disclosure as a whole.

We claim:

1. A medical device that is implantable within a patient, the medical device comprising:
a) a heart sensor for detecting a heartbeat of the patient and for generating a heart beat signal;
b) a processor for receiving the heart beat signal and for transitioning the medical device between an active state and an inactive state;
c) an electrical pulse output that is electrically connectible to a portion of the patient's heart; and
d) a power harvester that is adapted to harvest electric current from the patient's body and the power harvester is electrically connected to at least the pulse output and the processor so that the medical device is electrically self-sustaining when in an active state.

2. The medical device of claim 1, further comprising a surge sensor for detecting at least one of an electrical shock and an electric current surge that are greater than a pre-set level.

3. The medical device of claim 1, wherein the power harvester is electrically connectable to the pulse output by an electrical connection line, wherein the electrical connection line comprises a relay.

4. The medical device of claim 3, wherein the relay is actuatable to break the electrical connection between the power harvester and the pulse output.

5. The medical device of claim 1, wherein when the medical device is in the inactive state at least a portion of the harvested electrical current is directed to a ground.

6. The medical device of claim 1, wherein the processor comprises a counting mechanism for directing the harvested electrical current to the pulse output at a predetermined frequency when the device is in the active state.

7. The medical device of claim 4, wherein the predetermined frequency is suitable for stimulating a patient's heart.

8. The medical device of claim 1, further comprising a secondary power source.

9. The medical device of claim 1, wherein the medical device comprises a flexible substrate, wherein the heart sensor, the processor, the electrical pulse output and the power harvester are mounted on the flexible substrate.

10. A medical implant device comprising:
a) a heart pulse sensor implant adapted to be implanted into a live human body in cooperation with a heart within the body so as to detect a heartbeat of the heart;
b) a processor implant adapted to be implanted into the live human body and for cooperating with the heart pulse sensor implant to monitor the heartbeat of the heart and to detect when there is no heartbeat; and,
c) an energy harvesting implant, wherein the energy harvesting implant is adapted to be implanted in the live human body for electrically conductive coupling to an internal, electricity producing portion of the body so as to collect and transmit an electrical current derived therefrom so that the medical implant device is electrically self-sustaining.

11. The medical implant device of claim 10 further comprising an electrical pulse output device that is adapted to be electrically connected to a portion of the patient's heart.

12. The medical implant device of claim 10, further comprising a surge sensor that is adapted to detect at least one of an electrical shock and an electric current surge that are greater than a pre-set level.

13. The medical implant device of claim 12, wherein the pre-set level is suitable to stimulate the heart.

14. The medical implant device of claim 10, wherein the energy harvesting implant is electrically connected the electrical pulse output device of the medical implant device by an electrical connection line, wherein the electrical connection line comprises a relay and wherein the relay is actuatable to break the electrical connection between the energy harvesting implant and the electrical pulse output device.

15. The medical implant device of claim 10, wherein when the medical implant device is in the inactive state at least a portion of the harvested electrical current is directed to a ground.

16. The medical implant device of claim 10, wherein the processor implant comprises a counting mechanism for directing the harvested electrical current to the pulse output device at a predetermined frequency when the medical implant device is in the active state.

17. The medical implant device of claim 16, wherein the predetermined frequency is suitable for stimulating a patient's heart.

18. The medical implant device of claim 10, further comprising a secondary power source.

19. The medical implant device of claim 10, wherein the medical implant device comprises a flexible substrate, wherein the heart pulse sensor implant, the processor implant and the energy harvesting implant are mounted on the flexible substrate.

20. A method of using the medical implant device of claim 10 comprising steps of:
a) providing the medical implant device;
b) implanting the medical implant device;
c) establishing an electrical connection between the implanted medical implant device and a heart of a recipient of the implanted medical device implant; and
d) collecting electric current from the recipient so as to make the medical implant device electrically self-sustaining.
